(19) United States
(12) Patent Application Publication
(10) Pub. No.: US 2020/0146559 A1
(43) Pub. Date: May 14, 2020

(54) Elastic Photoelectric Sensor Module

(71) Applicant: Hangzhou Megasens Technologies Co., Ltd., Zhejiang (CN)

(72) Inventors: Haiquan Yuan, Zhejiang (CN); Congcong Zhou, Zhejiang (CN)

(21) Appl. No.: 16/678,572

(22) Filed: Nov. 8, 2019

(30) Foreign Application Priority Data
Nov. 8, 2018 (CN) 201811324683.0
Nov. 8, 2018 (CN) 201821835108.2

(51) Int. Cl. A61B 5/0205; A61B 5/1455; A61B 5/021; A61B 5/024

(52) U.S. Cl. CPC A61B 5/0205; A61B 5/14552; A61B 5/681; A61B 5/024; A61B 5/021

(57) ABSTRACT

The present invention discloses an elastic photoelectric sensor module, which is mainly used for monitoring of physiological parameters such as human heart rate, blood oxygen saturation and a blood pressure based on optical inspection. The module includes a sensor structural part (1), an elastic telescopic unit (2), a sensor window group (3), and a photoelectric sensor (4). The sensor structural part (1) provides structural support for the sensor device, and the elastic telescopic unit (2) provides elastic force for the device. The sensor window group (3) is a signal path, and the photoelectric sensor (4) is a basic signal detection unit.

### RELATED APPLICATIONS

This application claims priority under 35 U.S.C. § 119(b) to China, Application Nos. 201811324683.0 and 201821835108.2 both filed on Nov. 8, 2018. The entire teachings of the above applications are incorporated herein by reference.

### TECHNICAL FIELD

The present invention belongs to a health monitoring sensor, and particularly relates to monitoring of physiological parameters such as human heart rate, blood oxygen saturation and blood pressure based on optical inspection.

### BACKGROUND

Detection of human physiological parameters based on principle of photoelectric technology has been widely used in many aspects such as monitoring of critically ill patients in clinic, monitoring of intraoperative anesthesia, recovery of postoperative patients, screening and management of sleep apnea syndrome, and community medical monitoring. This technology is safe and reliable and can be performed continuously and non-invasively in real time. Continuous research on the principles and continuous improvement in measurement methods have promoted the development of detection technology of human heart rate, blood oxygen saturation, and blood pressure, and it is very meaningful to design an elastic photoelectric sensor module that can perform monitoring in various general health management scenarios. A typical scenario based on “Internet+Health Care” also requires a new generation of detection devices to have features of intelligentization, hardware miniaturization, and low power consumption. Real-time monitoring of multiple physiological parameters can be provided in a home-centered health management system and services can be provided for early disease screening, diagnosis, and personal health care based on new intelligent equipment.

### SUMMARY

In view of deficiencies in the present art, an object of this invention is to provide an elastic photoelectric sensor module, so that accurate measurement of physiological signals based on the principle of photoelectric detection is ensured when proper contact pressure is applied under comfortable wearing. The elastic photoelectric sensor module is adapted to detection of physiological parameters of different parts of human body to improve signal-to-noise ratio in the first place.

To achieve the above object, the present invention discloses an elastic photoelectric sensor module. The elastic photoelectric sensor module is mainly applied to a monitoring device for physiological parameters such as human heart rate, blood oxygen saturation, blood pressure based on optical principle, and can provide stable and reliable original pulse wave signal. The sensor module includes a sensor structural part, an elastic telescopic unit, a sensor window group, and a photoelectric sensor. The sensor structural part mainly provides structural support for the elastic photoelectric sensor module, the elastic telescopic unit provides elastic force for the elastic photoelectric sensor module, the sensor window group is a signal path, and the photoelectric sensor is a basic signal detection unit.

The elastic photoelectric sensor module in the present invention adopts materials such as metal and silica gel, and has the following features: 1. The module is simple to process and manufacture. 2. The module is sealed and has good insulation performance. 3. The module has high space utilization and is applicable to a miniaturized structure design.

Further, the elastic force provided by the elastic telescopic unit in the elastic photoelectric sensor module of the present invention is adjustable, and a spring is used, elastic potential energy is calculated by using formula:

$${\int }_{x}^{0} - kxdx = \frac{1}{2} {kx}^{2} ,$$

where k is elastic coefficient, and x is the deformation variable within the elastic limit of the spring. If a metal elastic piece is used, the elastic force is calculated by a formula:

$$F = \frac{b * {h}^{3} * e * δ}{4 * {l}^{3}} ,$$

where b is the width of the elastic piece, h is the thickness of the elastic piece, e is elastic coefficient, δ is deformation quantity, and l is the length of the elastic piece. One advantage of the adjustable elastic force is that the sensor module can be in better contact with a detected tissue under proper pressure, and comfort is improved at the same time.

Further, the sensor window group in the elastic photoelectric sensor module of the present invention is located directly above the elastic photoelectric sensor module, and the top of the sensor window is in contact with the human body. The sensor window group is formed by biocompatible glue such as silica gel to form a drop-shaped protrusion, to further ensure good contact.

Beneficial effects of the present invention are as follows: The present invention designs an elastic photoelectric sensor module, the module is simple to process and manufacture with good consistency, is sealed by glue during processing with good sealing performance and insulation performance, and has a small size, and is applicable to a miniaturized structure design. The telescopic unit is elastically adjustable, so that the photoelectric sensor is in close contact with the tissue under proper pressure, in which wearing comfort can be improved while ensuring signal quality. The module can be applied to use scenarios, in which the photoelectric sensor module is needed, such as long-time continuous monitoring of blood oxygenation saturation, heart rate and non-invasive continuous monitoring of blood pressure, and has a broad application prospect.

### BRIEF DESCRIPTION OF DRAWINGS

FIG. 1 is a basic block diagram according to an embodiment of the present invention;

FIG. 2 is a schematic diagram of a photoelectric sensor and a sensor window according to an embodiment of the present invention;

FIG. 3 is a schematic diagram of a structural part according to an embodiment of the present invention; and

FIG. 4 is a schematic diagram of the travel of a photoelectric sensor according to an embodiment of the present invention.

### DETAILED DESCRIPTION OF EMBODIMENTS

To make technical means and creative features implemented by the present invention, and achieved object and effects easy to understand, the present invention will be further described with reference to the accompanying drawings and specific embodiments.

With the development of smart wearable devices, the product form of wristbands and watches has gradually become clear. In addition to measuring daily activity data and recording trajectories, the pursuit of compactness and thinness is also a major trend in this field. In the field of wearable health and medical care, there are greater researches and application prospects to use new forms of devices to weaken properties of medical devices and ensure data accuracy at the same time. Therefore, a design of a miniaturized sensor module with high-performance and easy processing is especially valuable. Detection technology based on photoplethysmography (PPG) can implement real-time tracing of human pulse wave waveforms in different parts, and is used in the field of monitoring related physiological parameters that are based on pulse waves. In specific embodiments, a design of reflective sensor module with optimized sensor structure is applied, so that individual differences can be effectively reduced and device reliability can be improved.

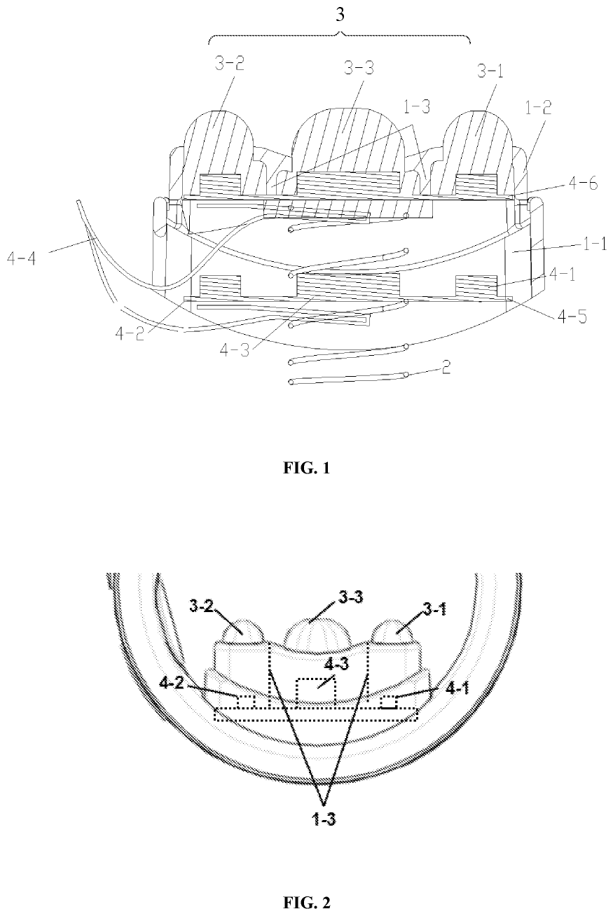

FIG. 1 is a basic block diagram according to an embodiment of the present invention. A sensor structural part 1 is included and provides structural support for an elastic photoelectric sensor module. The sensor structural part 1 includes a bottom structural part 1-1 and a movable top structural part 1-2. The bottom structural part 1-1 is used for fixing the photoelectric sensor module and an external circuit board, the top structural part 1-2 is used for supporting an overall structure of the sensor module, and a baffle 1-3 is used for prevent light in the photoelectric sensor from illuminating directly from light emitting sensor arrays 4-1, 4-2 directly to an photoelectric receiving tube 4-3. The bottom structural part 1-1 and the movable top structural part 1-2 are connected by barbs, and the top structural part 1-2 can be integrally retracted into the bottom structural part 1-1 when being pressed by an external force. An elastic telescopic unit 2 is made from an elastic material and structure such as a metal spring, a metal dome or silica gel, and provides elasticity for the photoelectric sensor module. A sensor window group 3 is located directly above the photoelectric sensor module, the sensor window group 3 includes any one or a combination of a light pipe, glass, a semi-permeable membrane and/or silica gel, and the top (a portion that is in contact with the human body) of the sensor window group (3) is formed by biocompatible glue such as silica gel to form a drop-shaped projection. The sensor window group 3 includes three sensor windows 3-1, 3-2, 3-3. The sensor windows 3-1, 3-2 are respectively located directly above the light emitting diode arrays 4-1, 4-2. The sensor window 3-3 is located directly above the photoelectric receiving tube 4-3. The sensor window group provides a path for a photoelectric signal, and at the same time, can provide good contact. The photoelectric sensor 4 includes the geometrically symmetric light emitting diode arrays 4-1, 4-2 and the photoelectric receiving tube 4-3, and belongs to a signal (photoelectric volume pulse wave) detection unit of the photoelectric sensor module. An electrical connection interface 4-4 of the photoelectric sensor is a connection interface of an external circuit, and implements input and output control of the signal. Under the joint action of the elastic telescopic unit 2 and the pressure of the external tissue, a travel of the photoelectric sensor 4 can start from the bottom 4-5 of the sensor module to the sensor module to the top 4-6 of the sensor module. The elastic photoelectric sensor module can be filled with an insulating material to form a sealed and insulating cavity, and the insulating material includes silica gel, insulating varnish and the like.

FIG. 2 is a schematic diagram of a photoelectric sensor and a sensor window according to an embodiment of the present invention. The sensor window group 3 is located directly above the photoelectric sensor module and has a drop-shaped protrusion. The sensor windows 3-1, 3-2 are respectively located directly above the light emitting diode arrays 4-1, 4-2. The sensor window 3-3 is located directly above the photoelectric receiving tube 4-3. The sensor window group provides a path for the optoelectronic signal, and at the same time can provide good contact. Between the light emitting diode arrays 4-1, 4-2 and the photoelectric receiving tube 4-3, the structural part 1-3 prevents light from transferring directly from the light emitting diode to the photoelectric receiving tube.

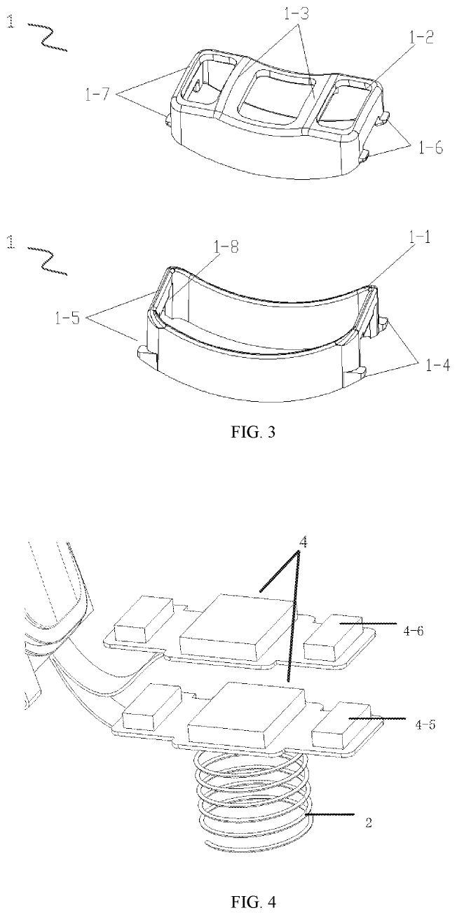

FIG. 3 is a schematic diagram of a structural part according to an embodiment of the present invention. A bottom structural part 1-1 is used for fixing the photoelectric sensor module and an external circuit board. Raised tentacles 1-4, 1-5 of the bottom structural part can be used for welding. A concave air rail 1-8 provides a moving rail for barbs 1-6, 1-7 of the top structural part 1-2. A baffle 1-3 is used for prevent light in the photoelectric sensor from directly illuminating directly from the light emitting sensor arrays 4-1, 4-2 to the photoelectric receiving tube 4-3. The bottom structural part 1-1 and the movable top structure part 1-2 are connected by the barbs, the top structural part 1-2 can be integrally retracted into the bottom structural part 1-1 when being pressed by an external force, and the outer part and the joint part of the structural part are filled with a material such as silica gel or insulating varnish to form a sealed and insulating cavity.

FIG. 4 is a schematic diagram of the travel of a photoelectric sensor according to an embodiment of the present invention. The photoelectric sensor 4 is operated by the elastic telescopic unit 2 and the pressure of the external tissue, and the travel can start from the bottom 4-5 of the sensor module to the sensor module to the top 4-6 of the sensor module.

Basic principles and main features of the present invention and advantages of the present invention are shown and described above. It should be understood by a person skilled in the art that the present invention is not limited by the foregoing embodiments, and only the principles of the present invention are described in the foregoing embodiments and the specification of the present invention. Without departing from the spirit and scope of the present invention, the present invention may further have various changes and modifications, and these changes and modifications shall fall within the scope of protection of the present invention. The scope of protection of the present invention is defined by the appended claims and their equivalents.

1. An elastic photoelectric sensor module, comprising: a sensor structural part (1), an elastic telescopic unit (2), a sensor window group (3), and a photoelectric sensor (4); wherein the sensor structural part (1) provides structural support for the elastic photoelectric sensor module; the elastic telescopic unit (2) provides an elastic force for the elastic photoelectric sensor module; the sensor window group (3) is a signal path, and is located at the top of the elastic photoelectric sensor module, and the top of the sensor window group (3) is in contact with human body; the photoelectric sensor (4) is a basic signal detection unit, which is capable of transmitting and receiving the photoelectric signal, and is located below the sensor window group (3).

2. The elastic photoelectric sensor module according to claim 1, wherein the material of the sensor structural part (1) comprises metal, plastic or silica gel to form a convex shaped structure or a triangular arrangement structure, and the sensor structural part (1) comprises a bottom structural part (1-1) and a movable top structural part (1-2) that are connected by a barb, and the top structural part (1-2) can be integrally retracted to the bottom structure (1-1) when being pressed by an external force.

3. The elastic photoelectric sensor module according to claim 1, wherein the elastic telescopic unit (2) is made from elastic material or structure comprising a metal spring, a metal dome, or a silica gel.

4. The elastic photoelectric sensor module according to claim 1, wherein the elastic telescopic unit (2) and the photoelectric sensor (4) form an integral structure, and the photoelectric sensor (4) comprises a first light emitting diode array (4-1) and a second light emitting diode array (4-2) that are geometrically symmetric, and a photoelectric receiving tube (4-3) located between the first light emitting diode array (4-1) and the second light emitting diode array (4-2).

5. The elastic photoelectric sensor module according to claim 2, wherein the elastic telescopic unit (2) and the photoelectric sensor (4) form an integral structure, and the photoelectric sensor (4) comprises a first light emitting diode array (4-1) and a second light emitting diode array (4-2) that are geometrically symmetric, and a photoelectric receiving tube (4-3) located between the first light emitting diode array (4-1) and the second light emitting diode array (4-2).

6. The elastic photoelectric sensor module according to claim 3, wherein the elastic telescopic unit (2) and the photoelectric sensor (4) form an integral structure, and the photoelectric sensor (4) comprises a first light emitting diode array (4-1) and a second light emitting diode array (4-2) that are geometrically symmetric, and a photoelectric receiving tube (4-3) located between the first light emitting diode array (4-1) and the second light emitting diode array (4-2).

7. The elastic photoelectric sensor module according to claim 1, wherein the sensor window group (3) comprises any one or a combination of a light pipe, glass, a semi-permeable membrane and/or silica gel, and the sensor window group (3) is formed by biocompatible glue to form a drop-shaped protrusion;
the sensor window group (3) comprises three sensor windows (3-1, 3-2, 3-3), wherein the first sensor window (3-1) and the second sensor window (3-2) are respectively located directly above the first light emitting diode array (4-1) and the second light emitting diode array (4-2), the third sensor window (3-3) is located directly above the photoelectric receiving tube (4-3).

8. The elastic photoelectric sensor module according to claim 1, wherein the elastic photoelectric sensor module is filled with an insulating material to form a sealed and insulating cavity.

9. The elastic photoelectric sensor module according to claim 2, wherein the elastic photoelectric sensor module is filled with an insulating material to form a sealed and insulating cavity.

10. The elastic photoelectric sensor module according to claim 3, wherein the elastic photoelectric sensor module is filled with an insulating material to form a sealed and insulating cavity.

11. The elastic photoelectric sensor module according to claim 7, wherein the elastic photoelectric sensor module is filled with an insulating material to form a sealed and insulating cavity.

12. The elastic photoelectric sensor module according to claim 4, wherein the elastic photoelectric sensor module is filled with an insulating material to form a sealed, insulating cavity.

13. The elastic photoelectric sensor module according to claim 5, wherein the elastic photoelectric sensor module is filled with an insulating material to form a sealed, insulating cavity.

14. The elastic photoelectric sensor module according to claim 6, wherein the elastic photoelectric sensor module is filled with an insulating material to form a sealed, insulating cavity.
